(19) United States
(12) Patent Application Publication
(10) Pub. No.: US 2005/0038248 A1
(43) Pub. Date: Feb. 17, 2005

(54) Chemotherapeutic agents

(76) Inventors: Scott Andrew Henderson, Victoria (AU); George Holan, Victoria (AU); Barry Ross Matthews, Victoria (AU)

Correspondence Address: FOLEY AND LARDNER;SUITE 500, 3000 K STREET NW, WASHINGTON, DC, 20007, US

(21) Appl. No.: 10/487,644

(22) PCT Filed: Aug. 29, 2002
(86) PCT No.: PCT/AU02/01180

(30) Foreign Application Priority Data
Aug. 30, 2001 (AU) PR 7383

(51) Int. Cl. C07D 43/02; C07D 41/02; C07C 255/49

(52) U.S. Cl. 544/405; 546/268.1; 562/465; 564/155; 558/410

(57) ABSTRACT

The invention provides 1,2-substituted cyclic compounds useful for treatment of diseases or disorders arising from abnormal or inappropriate cell proliferation, such as tumour growth, tumour metastasis and associated angiogenesis, as well as pharmaceutical compositions comprising these compounds and their use in methods of treatment.

### TECHNICAL FIELD

The present invention relates to novel compounds, processes for their production, and pharmaceutical compositions containing them as the active ingredient. In particular, this invention provides novel compounds useful for treating or preventing pathological states arising from abnormal or inappropriate cell proliferation—including angiogenesis, either alone or in conjunction with other treatments.

### BACKGROUND OF THE INVENTION

Neoplastic diseases are characterized by the uncontrolled proliferation of cells and are a major cause of death in mammals, including humans. Chemotherapeutic agents with various modes of action have been used to treat neoplastic disease, for example: antibiotics such as bleomycin and mitomycin; antimetabolites such as fluorouracil and methotrexate; microtubule polymerization inhibitors such as vincristine and colchicine; microtubule depolymerisation inhibitors such as paclitaxel and epothilone; and angiogenesis inhibitors such as angiostatin and neovastat.

Specifically, there is a need for chemotherapeutic agents for treatment of neoplastic diseases that are safe for therapeutic use and that exhibit selective toxicity with respect to the pathological condition. Furthermore, there is a need for chemotherapeutic agents with modified or improved profiles of activity.

### SUMMARY OF THE INVENTION

The present invention relates to a class of organic molecules that have antineoplastic activity. Such compounds are useful for the treatment of neoplastic diseases or neoplastic dependent disorders; illustrative of these are tumour growth, metastasis and associated angiogenesis. The present invention relates in particular to compounds that regulate and/or modulate abnormal or inappropriate cell proliferation, including any associated blood vessel growth (ie. angiogenesis).

Accordingly, the present invention provides 1,2-substituted cyclic compounds of Formula I: 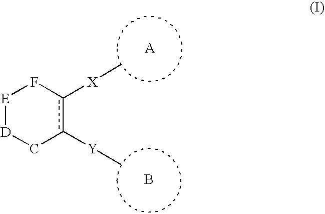
and pharmaceutically acceptable salts thereof,
wherein:
A and B are each independently selected from the group consisting of
alkyl, alkenyl, alkynyl, arylalkyl, heteroarylalkyl, cycloalkyl, heterocycloalkyl, aryl, and heteroaryl;
in which arylalkyl, heteroarylalkyl, cycloalkyl, heterocycloalkyl, aryl and heteroaryl groups may be connected with another ring through a single bond or fused with at least one other ring, and these rings optionally substituted at one or more positions with:
alkyl, alkoxy, aryl, aryloxy, arylalkyl, arylalkyloxy, cyano, halogen, nitro, oxo, thiono, or CHₙXₘ (where X is halogen, m is 1 to 3, and n is 3-m);
S(O)R, or S(O)₂R, (wherein R is selected from the group consisting of hydroxy, alkyl, alkoxy, aryl, aryloxy, arylalkyl, and arylalkyloxy);
C(O)R, NHC(O)R, or (CH₂)ₙC(O)OR, (wherein R is selected from the group consisting of hydrogen, hydroxy, alkyl, alkoxy, aryl, aryloxy, arylalkyl, and arylalkyloxy, and n is 0-11); S(O)₂OR, OR, SR, B(OR)₂, PR₃, P(O)(OR)₂, OP(O)(OR)₂, or ═NOR, (wherein R is selected from the group consisting of hydrogen, alkyl, aryl, and arylalkyl); or
NRR′, NRS(O)₂R′, SO₂NRR′, or CONRR′, (wherein R is selected from the group consisting of hydrogen, alkyl, aryl, and arylalkyl, and R′ is selected from the group consisting of hydrogen, hydroxy, alkyl, alkoxy, aryl, aryloxy, arylalkyl, and arylalkyloxy).
   The following limitations apply to A and B in Formula I:
(i) where A is alkyl, alkenyl, or alkynyl; B is an arylalkyl, heteroarylalkyl, cycloalkyl, heterocycloalkyl, aryl, or heteroaryl group; and
(ii) where B is alkyl, alkenyl, or alkynyl; A is an arylalkyl, heteroarylalkyl, cycloalkyl, heterocycloalkyl, aryl, or heteroaryl group.
   In Formula I:
   the dotted line bonds of the central ring indicate the possibility of a double bond or a delocalised aromatic bond;
C is CR¹, nitrogen, oxygen, or sulfur;
D is CR², nitrogen, oxygen, or sulfur;
E is CR³, nitrogen, oxygen, or sulfur;
F is CR⁴, nitrogen, oxygen, sulfur, or nothing;
   provided that at least one of C, D, E, or F is CR; and R¹, R², R³, R⁴ are each independently selected from:
   hydrogen, alkyl, alkenyl, alkynyl, alkoxy, aryl, aryloxy, arylalkyl, arylalkyloxy, cycloalkyl, cyano, halogen, heteroaryl, nitro, or CHₙXₘ (where X is halogen, m is 1 to 3, and n is 3-m);
   S(O)R, or S(O)₂R, (wherein R is selected from the group consisting of hydroxy, alkyl, alkoxy, aryl, aryloxy, arylalkyl, and arylalkyloxy);
   C(O)R, NHC(O)R, or (CH₂)ₙC(O)OR, (wherein R is selected from the group consisting of hydrogen, hydroxy, alkyl, alkoxy, aryl, aryloxy, arylalkyl, and arylalkyloxy, and n is 0-11);
   S(O)₂OR, OR, SR, B(OR)₂, PR₃, P(O)(OR)₂, OP(O)(OR)₂, or ═NOR, (wherein R is selected from the group consisting of hydrogen, alkyl, aryl, and arylalkyl); or
   NRR′, NRS(O)₂R′, SO₂NRR′, or CONRR′, (wherein R is selected from the group consisting of hydrogen, alkyl, aryl, and arylalkyl, and R′ is selected from the group consisting of hydrogen, hydroxy, alkyl, alkoxy, aryl, aryloxy, arylalkyl, and arylalkyloxy);
      or one of R¹ and R², or R² and R³, or R³ and R⁴ are taken together with the carbon atoms to which they are attached to form a carbocycle or heterocycle.
      Also in Formula I:
      X and Y are linker groups each selected independently from the group consisting of: SO₂NR, NRSO₂, C(O)NR, NRC(O), C(S)NR, NRC(S), NRC(O)O, NRC(S)S, C(O)O, OC(O), S(O)₂O, OSO₂, SO₂, OS(O), OSO₂NR, NRS(O)₂NR′, C(S)SSNR, NRSSC(S), P(O) (OR)NR′, NRP(O) (OR′), NRP(O)(OR′)O, CR═CR′, NRC(O)NR′, NR, C═NO—, —ON═C, C═N, N═C, N═N(→O)—, N(→O)═N, N═N, and a direct bond; where R and R′ are each selected independently from the group consisting of hydrogen, alkyl, alkenyl, alkynyl, cycloalkyl, aryl, arylalkyl, acyl, alkoxyacyl, aryloxyacyl, or aminoacyl (the above linker groups are shown with their left ends attached to the central ring and their right ends attached to the A or B ring).
      The following limitations apply to X and Y in Formula I:
(i) where X is NRSO₂, Y is not NRC(O), NRC(S), NR, NRC(O)O or NRC(O)NR;
(ii) where Y is NRSO₂, X is not NRC(O), NRC(S), NR, NRC(O)O or NRC(O)NR;
(iii) where X is a direct bond, Y is not a direct bond; and
(iv) where Y is a direct bond, X is not a direct bond.

The present invention also provides pharmaceutical compositions useful for the treatment of neoplastic diseases or neoplastic dependent disorders that comprise a therapeutically effective amount of a compound of Formula I, or a pharmaceutically acceptable salt thereof, and a pharmaceutically acceptable carrier or excipient.

The compositions of the present invention may be used for preventive or therapeutic treatment of diseases or disorders that involve uncontrolled proliferation of cells, such as tumour growth, tumour metastasis, and associated angiogenesis.

Accordingly, the present invention also provides a method for preventive and/or therapeutic treatment of a disease or disorder involving abnormal or inappropriate cell proliferation, which comprises administration of a therapeutically effective amount of a compound of Formula I, or a pharmaceutically acceptable salt thereof, to a human or other mammalian patient in need thereof. This treatment may be administered either alone or in conjunction with another preventative or therapeutic treatment of the disease or disorder.

Throughout this specification and the claims which follow, unless the context requires otherwise, the word “comprise”, and or variations such as “comprises” or “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

### DETAILED DESCRIPTION OF PREFERRED EMBODIMENTS

In the context of this description, the term “acyl” refers to a radical which is formed by removal of the hydroxy from a carboxylic acid (i.e., R—C[═O]—). Exemplary acyl groups include, acetyl, formyl, and propionyl. Such groups may be substituted or unsubstituted.

The term “alkenyl” refers to an unsubstituted or substituted, straight-chain or branched hydrocarbon radical having 2 to about 12 carbon atoms containing at least one carbon-carbon double bond, as exemplified by vinyl, propenyl, 2-butenyl, 3-butenyl, isobutenyl and 2-octenyl. The alkenyl group can be optionally substituted with one or more substituent. Suitable substituents include, but are not limited to: alkoxy, alkanoyl, alkanoyloxy, alkoxycarbonyl, amido, amino, aryloxy, aryl, azido, boronyl, carboxy, carboxaldehyde, cyano, cycloalkyl, cycloalkenyl, cycloalkoxy, halo, heteroaryl, heteroaryloxy, hydroxy, nitro, perfluoroalkyl, perfluoroalkoxy, thioalkoxy, trihalomethyl, phosphinyl, phosphonyl, sulfinyl, and sulfonyl.

The term “alkoxy” refers to an alkyl group attached to the parent molecular group through an oxygen atom, exemplified by substituted or unsubstituted methoxy, ethoxy, isopropyloxy, and tert-butyloxy.

The term “alkoxyacyl” refers to an acyl radical having an alkoxy substituent (i.e., —O—R), for example, —C(═O)-alkyl. Such groups may be substituted or unsubstituted.

The term “alkyl” refers to a straight-chain or branched saturated aliphatic hydrocarbon radical. Preferably the alkyl group has 1 to 12 carbons as exemplified by methyl, ethyl, propyl, isopropyl, butyl, isobutyl, sec-butyl, t-butyl, pentyl, i-pentyl, hexyl, heptyl, octyl and the like. The alkyl group can be optionally substituted with one or more substituent. Suitable substituents include, but are not limited to: alkoxy, alkanoyl, alkanoyloxy, alkoxycarbonyl, amido, amino, aryloxy, aryl, azido, boronyl, carboxy, carboxaldehyde, cyano, cycloalkyl, cycloalkenyl, cycloalkoxy, halo, heteroaryl, heteroaryloxy, hydroxy, nitro, perfluoroalkyl, perfluoroalkoxy, thioalkoxy, trihalomethyl, oxo, oxime, phosphinyl, phosphonyl, sulfinyl, and sulfonyl.

The term “alkynyl” refers to a straight-chain or branched hydrocarbon radical having two to about twelve carbon atoms containing at least one carbon-carbon triple bond, as exemplified by ethynyl, 2-propynyl, 2-butynyl, 2-pentynyl and 2-octynyl. The alkynyl group can be optionally substituted with one or more substituent. Suitable substituents include, but are not limited to: alkoxy, alkanoyl, alkanoyloxy, alkoxycarbonyl, amido, amino, aryloxy, aryl, azido, boronyl, carboxy, carboxaldehyde, cyano, cycloalkyl, cycloalkenyl, cycloalkoxy, halo, heteroaryl, heteroaryloxy, hydroxy, nitro, perfluoroalkyl, perfluoroalkoxy, thioalkoxy, trihalomethyl, oxo, oxime, phosphinyl, phosphonyl, sulfinyl, and sulfonyl.

The term “aminoacyl” refers to acyl groups having an amino substituent (i.e., —C(═O)—N); for example, —C(═O)—NH₂. The amino group of the aminoacyl moiety may be unsubstituted (i.e., primary amine) or may be substituted with one (i.e., secondary amine) or two (i.e., tertiary amine) alkyl groups.

The term “aryl” refers to mono- or bicyclic-carbocyclic ring system containing at least one aromatic ring. Examples of aryl groups include substituted or unsubstituted phenyl, naphthyl, 1,2-dihydronaphthyl, 1,2,3,4-tetrahydronaphthyl, fluorenyl, indanyl, azulenyl, and troponyl.

The term “arylalkyl” refers to an alkyl group with at least one aryl group attached, wherein “aryl” and “alkyl” are as defined as above. Examples of arylalkyl groups include benzyl, diphenylmethyl, triphenylmethyl, diphenylethyl, phenylethyl, phenylbutyl, and phenylpropyl. Such groups may be substituted or unsubstituted.

The term “arylalkyloxy” refers to O-arylalkyl groups wherein “aryl” and “alkyl” are as defined as above. Such groups may be substituted or unsubstituted.

The term “aryloxy” refers to an aryl group attached to the parent molecule via an oxygen atom. Such groups may be substituted or unsubstituted.

The term “aryloxyacyl” refers to an acyl radical having an aryloxy substituent. Such groups may be substituted or unsubstituted.

The term “cycloalkyl” refers to a cyclic hydrocarbon group of three to twelve carbon atoms. The cycloalkyl group can be optionally substituted with one or more substituent. Examples of cycloalkyl groups include substituted or unsubstituted cyclopropane, cyclobutane, cyclopentane, cyclohexane, cycloheptane, cyclohexanedione, cyclopentanedione, quinone, and tricyclododecane.

The term “heteroaryl” refers to a cyclic aromatic group having five or six ring atoms, wherein at least one ring atom is selected from the group consisting of nitrogen, oxygen, and sulfur, and the remaining ring atoms are carbon. The nitrogen atoms can be optionally quarternised, and the sulfur atoms can be optionally oxidized. Examples of heteroaryl groups include imidazole, furan, thiophene, pyrrole, isoxazole, pyrazole, isothiazole, triazole, tetrazole, pyridine, pyridazine, pyrimidine, pyrazine, and triazine. Such groups may be substituted or unsubstituted. The term “heteroaryl” also includes bicyclic or tricyclic rings, wherein the aforementioned heteroaryl ring is fused to one or two rings independently selected from the group consisting of aryl, cycloalkyl, heterocycloalkyl, and another heteroaryl ring. Examples include indole, benzo[b]furan, benzo[b]thiophene, benzimidazole, cinnoline, quinazoline, benzoxazole, purine, and pteridine. Such groups may be substituted or unsubstituted. The bicycic or tricyclic heteroaryl rings can be attached to the parent molecular group through either the heteroaryl group itself or the group to which it is fused.

The term “heteroarylalkyl” refers to an alkyl group with at least one heteroaryl group attached, wherein “alkyl” and “heteroaryl” are as defined above. Such groups may be substituted or unsubstituted.

The term “heterocycloalkyl” refers to a non-aromatic five-, six-, or seven-membered ring having between one and three heteroatoms independently selected from nitrogen, oxygen, and sulfur. Each five-membered ring has zero to one double bonds and each six-membered ring has zero to two double bonds. Examples of heterocycloalkyl groups include substituted or unsubstituted azetidinyl, pyrrolidinyl, piperidinyl, piperazinyl, morpholinyl, tetrahydrofuryl, and 1,2,3,4-tetrahydropyridinyl.

The term “pharmaceutically acceptable salt” refers to a salt of a compound of Formula I that is non-toxic and does not abrogate the biological activity and properties of the compound. Said salts can conveniently be obtained by treating either the basic forms of the compounds of Formula I with appropriate organic or inorganic acids, or by treating the acidic forms of the compounds of Formula I with appropriate organic or inorganic bases. Examples of the inorganic acids which may be employed to form pharmaceutically acceptable salts include such inorganic acids as hydrochloride, hydrobromide, hydroiodide, nitric, carbonic, sulfuric and phosphoric acid. Suitable pharmaceutically acceptable acid addition salts include but are not limited to the following: acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxy-ethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Water or oil-soluble or dispersible products are thereby obtained. Suitable pharmaceutically acceptable base addition salts include, for example, metallic salts made from aluminium, calcium, lithium, magnesium, potassium, sodium, and zinc, and organic salts made from N,N′-dibenzylethylenediamine, chloroprocaine, choline, diethanolamine, ethylenediamine, meglumine (N-methylglucamine) and procaine.

The compounds encompassed by Formula I may exhibit tautomerism or structural isomerism. Thus, while any given formula depicts one possible tautomeric or structural isomeric form, it should be understood that the invention encompasses any tautomeric or structural isomeric form, or mixtures thereof, possessing the ability to regulate and/or modulate abnormal or inappropriate cell proliferation and is not limited to any one tautomeric or structural isomeric form utilised within the formulae drawing.

The invention is further directed to solvated and unsolvated forms of the compounds of Formula I, and their pharmaceutically acceptable salts, having the ability to regulate and/or modulate abnormal or inappropriate cell proliferation including angiogenesis.

In one illustrative embodiment, the invention provides compounds Formula II: 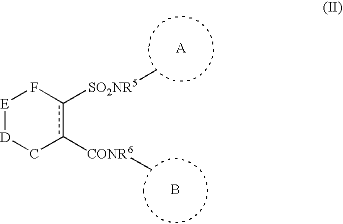
and pharmaceutically acceptable salts thereof, wherein A, B, C, D, E and F are as defined above; and wherein R⁵ and R⁶ are each independently selected from the group consisting of H, alkyl, and aryl.

In another embodiment, the compounds of the present invention have the formula: 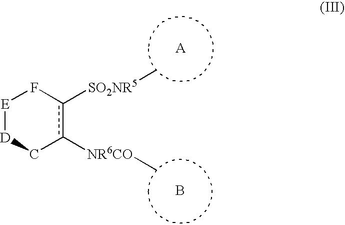
and pharmaceutically acceptable salts thereof, wherein A, B, C, D, E, F, R⁵, and R⁶ are as defined above.

In another embodiment, the compounds of the present invention have the formula: 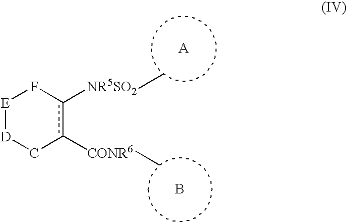
and pharmaceutically acceptable salts thereof, wherein A, B, C, D, E, F, R⁵, and R⁶ are as defined above.

In another embodiment, the compounds of the present invention have the formula: 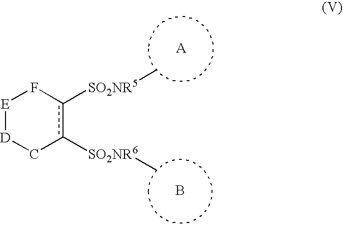
and pharmaceutically acceptable salts thereof, wherein A, B, C, D, E, F, R⁵, and R⁶ are as defined above.

In another embodiment, the compounds of the present invention have the formula: 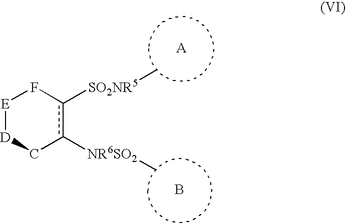
and pharmaceutically acceptable salts thereof, wherein A, B, C, D, E, F, R⁵, and R⁶, are as defined above.

In another embodiment, the compounds of the present invention have the formula: 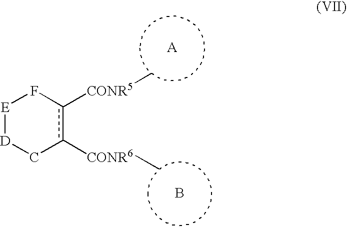
and pharmaceutically acceptable salts thereof, wherein A, B, C, D, E, F, R⁵, and R⁶ are as defined above.

In another embodiment, the compounds of the present invention have the formula: 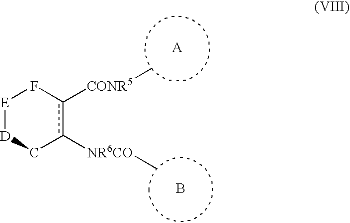
and pharmaceutically acceptable salts thereof, wherein A, B, C, D, E, F, R⁵, and R⁶ are as defined above.

In another embodiment, the compounds of the present invention have the formula: 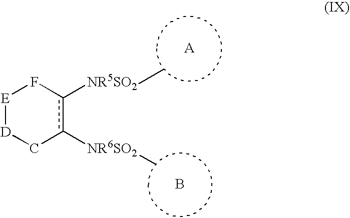
and pharmaceutically acceptable salts thereof, wherein A, B, C, D, E, F, R⁵, and R⁶ are as defined above.

In another embodiment, the compounds of the present invention have the formula: 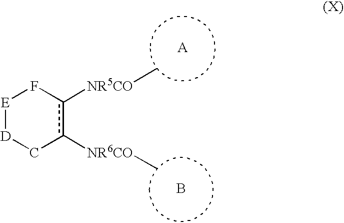
and pharmaceutically acceptable salts thereof, wherein A, B, C, D, E, F, R⁵, and R⁶ are as defined above.

In another embodiment, the compounds of the present invention have the formula: 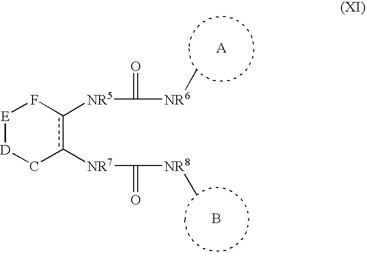
and pharmaceutically acceptable salts thereof, wherein A, B, C, D, E, F, R⁵, and R⁶ are as defined above, and wherein R⁷ and R⁸ are each independently selected from the group consisting of H, alkyl, and aryl.

In another embodiment, the compounds of the present invention have the formula: 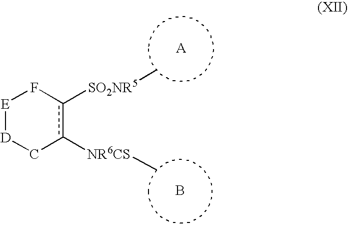
and pharmaceutically acceptable salts thereof, wherein A, B, C, D, E, F, R⁵, and R⁶ are as defined above.

In another embodiment, the compounds of the present invention have the formula: 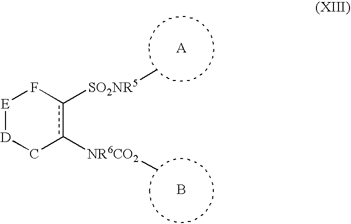
and pharmaceutically acceptable salts thereof, wherein A, B, C, D, E, F, R⁵, and R⁶ are as defined above.

In another embodiment, the compounds of the present invention have the formula: 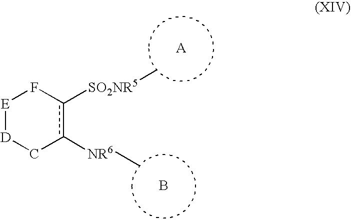
and pharmaceutically acceptable salts thereof, wherein A, B, C, D, E, F, R⁵, and R⁶ are as defined above.

In another embodiment, the compounds of the present invention have the formula: 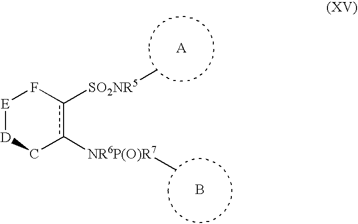
and pharmaceutically acceptable salts thereof, wherein A, B, C, D, E, F, R⁵, and R⁶ are as defined above; and wherein R⁷ is selected from the group consisting of H, alkyl, aryl, alkoxy, and aryloxy.

In another embodiment, the compounds of the present invention have the formula: 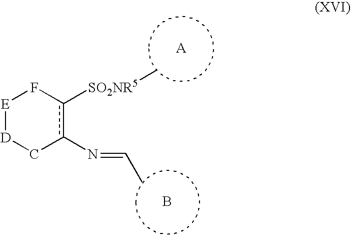
and pharmaceutically acceptable salts thereof, wherein A, B, C, D, E, F, and R⁵ are as defined above.

In another embodiment, the invention provides compounds having the formula: 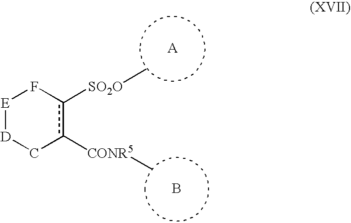
and pharmaceutically acceptable salts thereof, wherein A, B, C, D, E, F, and R⁵ are as defined above.

In another embodiment, the compounds of the present invention have the formula: 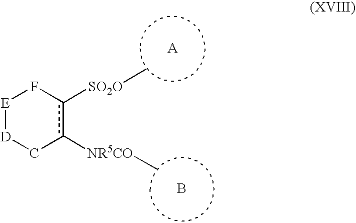
and pharmaceutically acceptable salts thereof, wherein A, B, C, D, E, F, and R⁵ are as defined above.

In another embodiment, the compounds of the present invention have the formula: 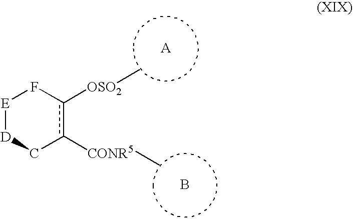
and pharmaceutically acceptable salts thereof, wherein A, B, C, D, E, F, and R⁵ are as defined above.

In another embodiment, the compounds of the present invention have the formula: 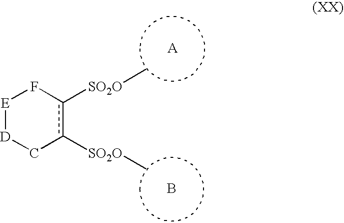
and pharmaceutically acceptable salts thereof, wherein A, B, C, D, E, and F are as defined above.

In another embodiment, the compounds of the present invention have the formula: 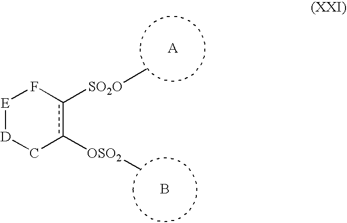
and pharmaceutically acceptable salts thereof, wherein A, B, C, D, E, and F are as defined above.

Preferably, in the compounds of Formulae I to XXI above, A and B are each selected independently from the group consisting of pyrrolidine, piperidine, piperazine, morphonline, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene, optionally substituted at one or more positions with alkyl, alkoxy, aryl, aryloxy, alkaryl, alkaryloxy, halogen, trihalomethyl, oxo, ═S, S(O)R, SO₂NRR′, S(O)₂OR, SR, B(OR)₂, PR₃, P(O)(OR)₂, OP(O)(OR)₂, NO₂, NRR′, N(O)R, OR, CN, C(O)R, NHC(O)R, (CH₂)ₙCO₂R, and CONRR′, wherein R and R′ are each independently selected from the group consisting of H, alkyl, alkoxy, aryl, aryloxy, arylalkyl, and arylalkyoxy; and n is 0-11.

Preferred compounds of the present invention include:
N-(2,6,-Diisopropylphenyl)-2-(2,6-diisopropylphenylsulfamoyl)-benzamide, N-phenyl-2-phenylsulfamoylbenzamide, N-[2-(4-methoxyphenylsulfamoyl)-phenyl]-isonicotinamide, N-[2-(4-methoxyphenylsulfamoyl)-phenyl]-4-nitrobenzamide, N-[2-(4-methoxyphenylsulfamoyl)-phenyl]-4-fluorobenzamide, N,N′-bis-(2,6-diisopropylphenyl)-phthalamide, 1-m-tolyl-3-[4-(3-m-tolyl-ureido)-pyridin-3-yl]-urea, 2-(4-methoxybenzenesulfonylamino)-N-pyridin-4-yl-benzamide, 2-(4-methoxybenzamido)-N-pyridin-4-ylbenzamide, [2-(4-methoxyphenylsulfamoyl)-phenyl]-carbamic acid tert-butyl ester, benzene-1,2-disulfonic acid 1-[(4-methoxyphenyl)-amide] 2-pyridin-4-yl amide, benzene-1,2-disulfonic acid bis-[(4-methoxyphenyl)-amide], thiophene-2-sulfonic acid [2-(4-methoxyphenylsulfamoyl)-phenyl]-amide, 1,2-bis(2,4,6-triisopropyl-N-phenyl)-benzenesulfonamide, 2-[benzyl-[(4-methoxyphenyl)-sulfamoyl]-N-pyridin-4-yl-benzamide, 2-[(4-methoxyphenyl)-sulfamoyl]-N-pyridin-4-yl-benzamide, 4-fluoro-N-[2-(3,4,5-trimethoxybenzene-sulfonylamino)-phenyl]-benzamide, 1H-pyrrole-2-carboxylic acid [2-(3,4,5-trimethoxybenzenesulfonylamino)-phenyl]-benzamide, and N-[2-(3,4,5-trimethoxybenzenesulfonylamino)-phenyl]-isonicotinamide.

The present invention relates to compounds capable of modulating/regulating and/or inhibiting cell proliferation for preventive and/or therapeutic treatment of pathological states, particularly neoplastic diseases or neoplastic dependent disorders. These diseases or disorders arising from abnormal or inappropriate cell proliferation include, for example, cancer and tumour metastasis.

More particularly, the present invention is directed to compounds that modulate/regulate and/or inhibit angiogenesis for preventive and/or therapeutic treatment of cancer, including astrocytoma, carcinoma, erythroblastoma, glioblastoma, leukemia, melanoma, meningioma, myoblastoma, and sarcoma. Indications may include, but are not limited to bladder cancers, blood cancers, bone cancers, brain cancers, breast cancers, colon cancers, gastric cancers, lung cancers, ovarian cancers, and pancreas cancers.

In view of the usefulness of the subject compounds in the preventive or therapeutic treatment of neoplastic diseases or neoplastic dependent disorders, the present invention provides a method for preventative and/or therapeutic treatment of a human or other mammal suffering from such a disease or disorder, said method comprising administration to said human or other mammal of a therapeutically effective amount of a compound of Formula I, or a pharmaceutically acceptable salt thereof. This treatment may be administered either alone or in conjunction with another preventative or therapeutic treatment of the disease or disorder.

In another aspect, the present invention also provides the use of a compound of Formula I, or a pharmaceutically acceptable salt thereof, in the manufacture of a composition for preventative and/or therapeutic treatment of a disease or disorder arising from abnormal or inappropriate cell proliferation.

In view of their useful pharmacological properties, the subject compounds may be formulated into various pharmaceutical forms for administration purposes. To prepare the pharmaceutical compositions of this invention, an effective amount of a particular compound, which may be in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or parenterally. Alternatively, a compound of the present invention may be administered as a pharmaceutical composition containing the compound of interest in combination with one or more pharmaceutically acceptable excipients. A “pharmaceutically acceptable” carrier or excipient refers to a non-toxic solid, semi-solid or liquid filler, diluent, encapsulating material or formulation auxiliary of any type. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelation and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral compositions, the carrier will usually comprise sterile water, at least in large part, though other ingredients, for example to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment. It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

When used in the preventative or therapeutic treatments described herein, a therapeutically effective amount of a compound of the present invention may be employed in pure form or, where such forms exist, in pharmaceutically acceptable salt form. By a “therapeutically effective amount” of the compound of the invention is meant a sufficient amount of the compound for preventative or therapeutic treatment of a neoplastic disease or neoplastic dependent disorder, (for example, to limit tumor growth, slow or block tumor metastasis, or inhibit angiogenesis) at a reasonable benefit/risk ratio applicable to any preventive or therapeutic medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disease or disorder being treated and the severity of the disease or disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

In light of the present description, and the results detailed herein, a person familiar with the pharmaceutical testing will understand the routine nature of determining a therapeutically effective amount of a compound of the invention. Thus, determining a therapeutically effective amount is well within the purview of the skilled clinician, and will depend on the exact identity of the active compound and particular patient characteristics, inter alia. General guidance can be found, for example, in the publications of the International Conference on Harmonisation.

Such a determination specifically will depend on such factors as the toxicity and efficacy profile of a given, active compound.

In an initial clinical trial, a patient in need of treatment or a normal volunteer typically is administered an active compound at a specific dose, usually low, at specified intervals for a period of time. In the absence of adverse effects, as determined by the clinician, this procedure may be repeated with successively higher doses of active compound. In this way, potentially toxic side-effects and parameters, such as bioavailability, may be determined using methods readily known in the art. Some typical pre-clinical and clinical parameters that are monitored are found in Remington's Pharmaceutical Sciences, chapters 27-28, pages 484-528 (Mack Publishing Company, 1990). With the results of the toxicology studies in mind, clinical trials for efficacy are undertaken.

In general, it is contemplated that an effective amount of a compound of the invention will be from 10⁻⁵ mg/kg to 100 mg/kg body weight, and in particular from 0.001 mg/kg to 10 mg/kg body weight. It may be appropriate to administer the required dose as two, three, four or more sub-doses at appropriate intervals throughout the day. Said sub-doses may be formulated as unit dosage forms, for example, containing 0.001 to 500 mg, and in particular 0.01 mg to 200 mg of active ingredient per unit dosage form.

The compounds of the present invention may be synthesised by known techniques. A general strategy for the synthesis of compounds of Formula I is to form linker X by reacting a 1,2-substituted cyclic compound with the appropriate compound to form component A. Linker Y can then be introduced by further reaction with a suitably substituted compound to form component B of the desired product. This approach is general and applicable to any combination of X and Y by the appropriate choice of starting materials, whether commercially available or prepared from by known methods.

Compounds where X is NR⁵SO₂ and Y is CONR⁶ may be prepared by reacting anthranilic acid with a sulfonyl chloride to form a sulfonamide bond, treatment with thionyl chloride to form the ortho-acid chloride and coupling with an amine or aniline to give the desired product. For example, compounds of Formula I where X is SO₂NR⁵ and Y is NR⁶SO₂ may be prepared by reacting an amine or aniline and 2-nitrobenzenesulfonyl chloride to form a sulfonamide bond, followed by reduction of the nitro group to an ortho-aniline and coupling with the a sulfonyl chloride to give the desired product. Compounds of Formula I where X is NR⁵SO₂ and Y is NR⁶SO₂ may be prepared by reacting a 2-nitroaniline with a sulfonyl chloride to form a sulfonamide bond, followed by reduction of the nitro group to an ortho-aniline and coupling with a second sulfonyl chloride to give the desired product. Compounds of Formula I where X is NR⁵CO and Y is NR⁶CO may be prepared, for example, by treating 1,2-phenylenediamine sequentially with two acid chlorides. Compounds of Formula I where X is SO₂NR⁵ and Y is NR⁶ may be prepared, for example, by reacting 2-bromobenzene-sulfonyl chloride with an amine or aniline, followed by palladium catalysed coupling to an amine or aniline. (J. F. Hartwig, et al. Journal of Organic Chemistry. 1999, volume 64, pages 5575-5580). Compounds of Formula I where X is SO₂NR⁵ and Y is NR⁶CS may be prepared, for example, by treating the corresponding compounds where Y is NR⁶CO with Lawesson's reagent to convert the amide carbonyl to a thiocarbonyl. (B. Yde et al. Tetrahedron. 1984. volume 40(11), pages 2047-2052). Compounds of Formula I where X is SO₂NR⁵ and Y is NR⁶P(O)R⁷ may be prepared, for example, by reacting the appropriate 2-sulfonamide substituted aniline with methylphenylphosphinoyl chloride. (C. S. Gibson and J. D. Johnson. Journal of the Chemical Society. 1928. pages 92-99). Compounds of Formula I where X is SO₂O and Y is CONR⁵ may be prepared, for example analogously to example 1, by reacting 2-sulfobenzoic acid with an alcohol or phenol, forming the acid chloride by treatment with thionyl chloride and reacting with an amine or aniline. Compounds of Formula I where X is SO₂NR⁵ and Y is N═CH may be prepared, for example, by treating 2-nitrobenzenesulfonyl chloride with an amine or aniline to form a sulfonamide bond, followed by reduction of the nitro group to an ortho-aniline and coupling with an aldehyde to give the desired product. Compounds of the Formula I where X is SO₂O and Y is NR⁵CO may be prepared, for example, analogously to example 3 but using an alcohol or phenol instead of 4-methoxyaniline. Compounds of the Formula I where X is SO₂O and Y is SO₂O may be prepared, for example, analogously to example 11 but using alcohols or phenols instead of the aniline derivatives. Compounds of the Formula I where X is SO₂O and Y is SO₂O may be prepared, for example, by treating 2-hydroxybenzene sulfonic acid with the desired sulfonyl chloride, formation of the sulfonyl chloride by treatment with thionyl chloride, and reaction with the desired alcohol or phenol. Compounds of the Formula I where X is OSO₂ and Y is CONR⁵ may be prepared, for example, by treating salicylic acid with the desired sulfonyl chloride, formation of the sulfonyl chloride by treatment with thionyl chloride, and reaction with the desired amine or aniline. Compounds of the Formula I where X is a direct bond and Y is OC(O) may be prepared, for example, by condensing phenylphenol with the desired carboxylic acid to form the ester linkage. Compounds of the Formula I where X is CR═CR′ and Y is C═N may be prepared, for example, by treating 2-stilbenecarboxaldehyde with the desired amine or aniline to form the imine linkage. Compounds of the Formula I where X is N═N and Y is C(O)O may be prepared, for example, by an aniline with hydrogen peroxide in acetic acid to form the nitroso compound (R. R. Holmes and R. P. Bayer. Journal of the American Chemical Society. 1960. vol 82. page 3454). Reaction of the nitroso compound with the anthranilic acid in acetic acid forms the azo linkage (J. March. Advanced Organic Chemistry. 4^{th} edition. page 638). Esterification with the desired alcohol or phenol to give the desired product. Compounds of Formula I where X or Y is N(→O)=N or N═N(→O) may be prepared by oxidation of the corresponding azo compound with hydrogen peroxide. Compounds of the Formula I where X is C═NO— and Y is OC(O) may be prepared, for example, by treating salicylaldehyde with hydroxylamine to give an oxime which when treated with an alkyl halide forms X, followed by condensation with a carboxylic acid to give the desired product. Compounds of the Formula I where X is SO₂ and Y is NRSO₂ may be prepared, for example, by treating 2-nitrobenzene-sulfonyl chloride with benzene under Friedel-Crafts conditions to form X, followed by reduction of the nitro group to give an ortho-aniline and coupling with a sulfonyl chloride to give the desired product. Compounds of Formula I where X or Y is —ON═C, may be prepared, for example, by treating the appropriate aryl bromide for the central ring with an oxime. Compounds of Formula I where X or Y is NRC(S)S, may be prepared, for example, by reacting a thiol with carbon disulfide followed by the appropriate aniline for the central ring. Compounds of Formula I where X or Y is OS(O), may be prepared, for example, by treating the appropriate aryl diazonium salt for the central ring with a sulfone. Compounds of Formula I where X or Y is P(O)(OR)NR′, may be prepared, for example, by treating the appropriate substituted phenyl phosphonic acid mono ester with the desired aniline in the presence of dicyclohexylcarbodiimide. Compounds of Formula I where X or Y is NRP(O)(OR′), may be prepared, for example, by treating the appropriate O-aryl phenylphosphonochloridate with an aniline to give the phosphonamidate. Compounds of Formula I where X or Y is NRP(O)(OR′)O may be prepared, for example, by treating a 1,2-phenylenediamine with an arylphosphoric acid dichloride to form a phosphol-2-oxide which reacts with water to give the phosphoric acid diamide ester. Compounds of Formula I where X or Y is OS(O)₂NR, may be prepared, for example, by reacting 1,3-disubstituted sulfonic acid diamides with the appropriate phenol. Compounds of Formula I where X or Y is NRS(O)₂NR′, may be prepared, for example, by the reaction of an aryl N-acetyl N-(chlorosulfonyl)-amide with the desired aniline followed by alkaline hydrolysis (D. L. Forster et al. Journal of the Chemical Society Section C. 1971. page 993).

The following examples are included by way of illustration, not limitation of the invention.

### EXAMPLE 1

N-(2,6,-Diisopropylphenyl)-2-(2,6-diisopropylphenylsulfamoyl)-benzamide

2-Sulfobenzoic acid ammonium salt (1.8 g, 8.3 mmol) was dissolved in water (10 mL) and ion-exchanged using an IR-120 (acid form) ion-exchange column to give 2-sulfobenzoic acid as a white solid (1.6 g, 95%). 2-Sulfobenzoic acid (1.2 g, 5.9 mmol) was dissolved in thionyl chloride (20 mL) and DMF (0.2 mL) and heated at reflux for 15 h. On cooling the solvent was removed in vacuo to give 2-chlorosulfonylbenzoyl chloride as a clear yellow oil (1.4 g, 98%).

2,6-Diisopropylaniline (1.66 mL, 8.8 mmol) and triethylamine (1.23 mL, 8.8 mmol) were added dropwise to a solution of 2-chlorosulfonylbenzoyl chloride (1.0 g, 4.2 mmol) in chloroform (25 mL). The mixture was stirred under nitrogen at room temperature for 15 h. The solvent was removed in vacuo and the residue chromatographed on silica gel using ethyl acetate as eluent to give N-(2,6,-diisopropylphenyl)-2-(2,6-diisopropylphenylsulfamoyl)-benzamide as an off-white solid (1.1 g, 48%). ¹H NMR (200 MHz, CDCl₃) δ 0.85 (d, J=6.7 Hz, 6H), 0.92 (d, J=6.9 Hz, 6H), 1.25 (d, J=6.6 Hz, 6H), 1.33 (d, J=7.0 Hz, 6H), 3.05 (quin, J=6.5 Hz, 2H), 3.38-3.72 (m, 2H), 6.79 (d, J=7.8 Hz, 1H), 7.04-7.6 (m, 6H), 7.36-7.60 (m, 4H), 8.42 (d, J=7.9 Hz, 1H), 12.73 (br s, 1H); ¹³C NMR (63 MHz, CDCl₃) δ 21.9, 22.4, 24.7, 25.7, 28.3, 28.8, 121.5, 124.7, 125.3, 127.0, 128.9, 129.1, 129.1, 129.9, 131.4, 133.0, 144.3, 146.4, 146.9, 163.7; MS (APCI−) m/z 519 (M−H); MS (APCI+) m/z 521 (M+H).

### EXAMPLE 2

N-Phenyl-2-phenylsulfamoylbenzamide

2-Chlorosulfonylbenzoyl chloride (0.81 g, 3.4 mmol) and aniline (1.23 mL, 13.5 mmol) were dissolved in toluene (30 mL) and heated at reflux for 48 h. The solvent was removed under reduced pressure and the residue chromatographed on silica gel using ethyl acetate/petroleum spirit (40-60° C.) (1:3) as eluent to give N-phenyl-2-phenylsulfamoylbenzamide as an off-white solid (0.15 g, 13%). ¹H NMR (200 MHz, CDCl₃) δ 6.96-7.28 (m, 10H), 7.48-7.67 (m, 4H), 8.30 (br s, 1H), 8.95 (br s, 1H); MS ((APCI−)) m/z 351 (M−H); MS (APCI+) m/z 353 (M−H).

### EXAMPLE 3

N-[2-(4-Methoxyphenylsulfamoyl)-phenyl]-isonicotinamide

4-Methoxyaniline (11.0 g, 8.12 mmol) was added to a stirred mixture of 2-nitrobenzenesulfonyl chloride (1.98 g, 8.93 mmol) and triethylamine (1.3 mL, 9.33 mmol) in dichloromethane (100 mL). The mixture was stirred at rt under nitrogen for 16 h. Reaction mixture was poured into water (100 mL), the organic layer separated, dried (MgSO₄), and concentrated under reduced pressure to give N-(4-methoxyphenyl)-2-nitrobenzenesulfonamide as a tan solid (2.45 g, 98%).

A suspension of N-(4-methoxyphenyl)-2-nitrobenzenesulfonamide (0.21 g, 0.66 mmol) and 10% palladium on carbon (20 mg) in ethanol (20 mL) and acetic acid (0.2 mL) was vigorously stirred under a hydrogen atmosphere for 18 h at rt. The mixture was filtered through a celite plug and washed with ethanol. The solvent was removed under reduced pressure to give 2-amino-N-(4-methoxyphenyl)benzenesulfonamide as a white solid (0.18 g, 97%).

Dimethylformamide (0.1 mL) was added dropwise to a chilled (ice-water bath) solution of isonicotinic acid (58 mg, 0.45 mmol) and oxalyl chloride (40 μL, 0.45 mmol) in dichloromethane (2 mL) under nitrogen. Stirring was continued at ambient temperature for 40 min. A solution of 2-amino-N-(4-methoxyphenyl)benzenesulfonamide (84 mg, 0.30 mmol) in dichloromethane (2 mL) was added to the reaction flask and stirring continued for 18 h. A precipitate formed and was collected by filtration to give N-[2-(4-methoxyphenylsulfamoyl)-phenyl]-isonicotinamide as a white solid (00.10 g, 86%). ¹H NMR (200 MHz, d₆-DMSO) δ 3.58 (s, 3H), 6.65 (d, J=9.0 Hz, 2H), 6.85 (d, J=9.0 Hz, 2H), 7.27-7.36 (m, 1H), 7.63-7.75 (m, 4H), 8.27 (br d, J=8.2 Hz, 1H), 8.81-8.85 (m, 2H), 10.07 (br s, 1H), 10.14 (br s, 1H); ¹³C NMR (50.3 MHz, d₆-DMSO) δ 55.0, 114.3, 120.9, 123.3, 124.7, 125.5, 128.3, 128.5, 129.2, 133.9, 135.3, 140.7, 150.5, 157.3, 162.9; MS ((APCI−)) m/z 382 (M-H); MS (APCI+) m/z 384 (M−H).

### EXAMPLE 4

N-[2-(4-Methoxyphenylsulfamoyl)-phenyl]-4-nitrobenzamide

Dimethylformamide (0.1 mL) was added dropwise to a chilled (ice-water bath) solution of 4-nitrobenzoic acid (74 mg, 0.44 mmol) and oxalyl chloride (40 μL, 0.45 mmol) in dichloromethane (2 mL) under nitrogen. Stirring was continued at ambient temperature for 40 min. A solution of 2-amino-N-(4-methoxyphenyl)benzenesulfonamide (82 mg, 0.30 mmol, preparation is described in Example 3) in dichloromethane (2 mL) was added to the reaction flask and stirring continued for 18 h. A precipitate formed and was collected by filtration to give N-[2-(4-methoxyphenylsulfamoyl)-phenyl]-4-nitrobenzamide as a white solid (0.72 g, 57%). ¹H NMR (200 MHz, d₄-MeOD) δ 3.70 (s, 3H), 6.72-6.80 (m, 2H), 6.95-7.14 (m, 4H), 7.37-7.49 (m, 1H), 7.52-7.59 (m, 1H), 8.20-8.26 (m, 3H), 8.28-8.34 (m, 3H); MS ((APCI−)) m/z 426 (M−H).

### EXAMPLE 5

N-[2-(4-Methoxyphenylsulfamoyl)-phenyl]-4-fluorobenzamide

Dimethylformamide (0.15 mL) was added dropwise to a chilled (ice-water bath) solution of 4-fluorobenzoic acid (150 mg, 1.08 mmol) and oxalyl chloride (95 μL, 1.08 mmol) in dichloromethane (5 mL) under nitrogen. Stirring was continued at ambient temperature for 40 min. A solution of 2-amino-N-(4-methoxyphenyl)benzene-sulfonamide (0.2 g, 0.72 mmol, preparation described in Example 3) and triethylamine (0.15 mL, 1.08 mmol) in dichloromethane (5 mL) was added to the reaction flask and stirring continued for 18 h. The reaction mixture was partitioned between ethyl acetate and brine. The organic layer was separated and the aqueous layer extracted trice with ethyl acetate. The combined organic extracts were dried (MgSO₄), filtered and concentrated under reduced pressure to give crude product. Purification by flash chromatography on silica gel (2% methanol in dichloromethane eluent) gave N-[2-(4-methoxyphenylsulfamoyl)-phenyl]-4-fluoro-benzamide as a white solid (0.19 g, 65%). ¹H NMR (200 MHz, d₄-MeOD) δ 3.75 (s, 3H), 6.70-6.75 (m, 1H), 6.85-6.90 (m, 2H), 6.95-7.05 (m, 1H), 7.18-7.25 (m, 1H), 7.50 (d, J=10.0 Hz, 2H), 7.85-7.90 (m, 1H), 8.45 (d, J=8.0 Hz, 2H), 9.00 (d, J=8.0 Hz, 2H); ¹³C NMR (50.3 MHz, d₆-DMSO) δ 55.0, 114.3, 115.5, 115.9, 122.6, 124.0, 125.5, 127.5, 128.6, 129.1, 129.8, 130.0, 130.3, 133.9, 135.9, 157.3, 161.9, 163.3, 166.8; MS ((APCI−)) m/z 399 (M−H).

### EXAMPLE 6

N,N′-Bis-(2,6-diisopropylphenyl)-phthalamide

Triethylamine (1.8 mL, 12.9 mmol) and 2,6-diisopropylaniline (90%, 2.7 mL, 12.9 mmol) were added to a solution of phthaloyl dichloride (1.24 g, 6.1 mmol) in dichloromethane (20 mL). The mixture was stirred at room temperature for 15 h. Dichloromethane (50 mL) was added and the mixture partitioned between dichloromethane and water (50 mL). The organics were separated and concentrated in vacuo and the residue chromatographed on silica gel using ethyl acetate/petroleum spirit (40-60° C.) (1:9) as eluent to give N,N′-bis-(2,6-diisopropylphenyl)-phthalamide as a white solid (0.09 g, 3%). ¹H NMR (200 MHz, CDCl₃) δ 1.22 (d, J=6.8 Hz, 24H), 3.27 (quin, J=6.9 Hz, 4H), 7.15-7.38 (m, 6H), 7.60-7.65 (m, 2H), 7.93-7.99 (m, 2H), 8.16 (br s, 2H); ¹³C NMR (50 MHz, CDCl₃) δ 23.7, 28.7, 123.4, 128.4, 129.3, 130.9, 131.0, 135.8, 146.2, 168.1; MS ((APCI−)) m/z 483 (M−H).

### EXAMPLE 7

1-m-Tolyl-3-[4-(3-m-tolyl-ureido)-pyridin-3-yl]-urea

m-Tolylisocyanate (0.6 mL, 4.7 mmol) was added dropwise to a stirred suspension of 3,4-diaminopyridine (0.5 g, 4.58 mmol) in benzene (5 mL) at room temperature over a period of 30 min. The mixture was heated at reflux for 4 h, allowed to cool and left to stand overnight under a nitrogen atmosphere. The precipiate was collected by filtration and washed with benzene. The crude product was purified by flash chromatography on silica gel (10% methanol in dichloromethane as eluent) to give 1-m-tolyl-3-[4-(3-m-tolyl-ureido)-pyridin-3-yl]-urea as a white solid (0.18 g, 10%). ¹H NMR (200 MHz, d₄-MeOD/CDCl₃) δ 2.20 (s, 3H), 2.23 (s, 3H), 6.70-6.81 (m, 2H), 7.04-7.18 (m, 6H), 7.98 (d, J=6.0 Hz, 1H), 8.12 (d, J=6.0 Hz, 1H), 8.18 (br s, 1H); ¹³C NMR (50.3 MHz, d₆-DMSO) δ 122.6, 123.1, 123.4, 128.5, 128.7, 137.8, 138.0, 139.0, 139.6, 142.1, 146.8, 148.1, 151.9, 153.7; MS (APCI+) m/z 376 (M+H).

### EXAMPLE 8

2-(4-Methoxybenzenesulfonylamino)-N-pyridin-4-yl-benzamide

A suspension of anthranilic acid (2.0 g, 14.6 mmol), dicyclohexylcarbodiimide (4.6 g, 22.3 mmol), 4-dimethylaminopyridine (10 mg), and 4-aminnopyridine (1.65 g, 17.5 mmol) in N,N-dimethylformamide (100 mL) was stirred at room temperature under a nitrogen atmosphere for 16 h. The solvent was removed in vacuo and the residue chromatographed on silica gel (5% methanol in dichloromethane as eluent) to give a mixture of desired product and dicyclohexylurea. The crude product was purified by flash chromatography on silica gel (dichloromethane as eluent) to give 2-amino-N-pyridin-4-ylbenzamide as a white solid (0.24 g, 8%)

A solution of the sulfonyl chloride (0.27 g, 1.3 mmol) in dichloromethane (3 mL) was added dropwise to a solution of 2-amino-N-pyridin-4-ylbenzamide (0.25 g, 1.2 mmol) and triethylamine (0.2 mL, 1.4 mmol) in dichloromethane (5 mL) and stirred under a nitrogen atmosphere for 18 h. The reaction mixture was partitioned between dichloromethane and brine. The organic layer was separated and the aqueous layer extracted trice with ethyl acetate. The combined organic extracts were dried (MgSO₄), filtered and concentrated under reduced pressure to give crude product. Purification by flash chromatography on silica gel (dichloromethane as eluent) gave 2-(4-methoxybenzene-sulfonylamino)-N-pyridin-4-yl-benzamide as a light cream solid (45 mg, 10%). ¹H NMR (200 MHz, CDCl₃) δ 3.60 (s, 3H), 6.66-6.72 (m, 2H), 7.13-7.24 (m, 2H), 7.44-7.54 (m, 1H), 7.57-7.77 (m, 5H), 8.09 (dd, J=8.0, 1.5 Hz, 1H), 8.74 (dd, J=8.5, 1.0 Hz, 1H), 10.32 (br s, 1H), 11.72 (br s, 1H)

### EXAMPLE 9

2-(4-Methoxybenzamido)-N-pyridin-4-ylbenzamide

A suspension of p-anisic acid (0.5 g, 3.3 mmol) and thionyl chloride (6 mL, 82.3 mmol) was heated at reflux, under an atmosphere of nitrogen, for 16 h. Solvent was removed in vacuo to give 4-methoxybenzoyl chloride as a fawn oil. The acid chloride and N-(2-aminophenyl)isonicotinamide (0.1 g, 0.46 mmol, preparation is described in Example 8) were dissolved in dichloromethane (8 mL) and triethylamine (100 μL, 0.68 mmol). The mixture stirred at room temperature for 16 h. The reaction mixture was washed with 5% sodium bicarbonate solution, the aqueous layer separated and extracted with dichloromethane a further three times. The combined organics were dried (MgSO₄) and concentrated under reduced pressure to give the crude product as a tan oil. Purification by flash chromatography on silica gel (dichloromethane as eluent) gave 2-(4-methoxy-benzamido)-N-pyridin-4-ylbenzamide as a cream solid (45 mg, 30%). ¹H NMR (200 MHz, d₄-MeOD) δ 3.76 (s, 3H), 6.81-6.89 (m, 2H), 7.42-7.59 (m, 5H), 7.76-7.87 (m, 1H); MS (APCI+) m/z 348 (M+H).

### EXAMPLE 10

[2-(4-Methoxyphenylsulfamoyl)-phenyl]-carbamic acid tert-butyl ester

N-t-Butoxycarbonyl anhydride (0.2 g, 0.92 mmol) was added to a stirred solution of 2-amino-N-(4-methoxyphenyl)benzenesulfonamide (0.21 g, 0.75 mmol, preparation is described in Example 3), triethylamine (0.3 mL, 2.15 mmol), and N,N-dimethylaminopyridine (10 mg) in THF (8 mL). Mixture was stirred at rt for 18 h. The solvent was removed in vacuo and the residue portioned between dichloromethane and 0.5 M sodium bicarbonate solution. The aqueous layer was extracted with dichloromethane twice and the combined organics washed twice with saturated citric acid solution, dried (MgSO₄), and concentrated under reduced pressure. Purification by flash chromatography on silica gel (dichloromethane as eluent) gave [2-(4-methoxyphenylsulfamoyl)-phenyl]-carbamic acid tert-butyl ester as a light yellow oil (0.2 g, 81%). ¹H NMR (200 MHz, CDCl₃) δ 1.34 (s, 9H), 3.83 (s, 3H), 4.76 (br s, 1H), 6.70-6.82 (m, 2H), 6.86-6.94 (m, 2H), 7.22-7.39 (m, 3H), 7.75 (dd, J=8.0, 1.6 Hz, 1H); MS (APCI+) m/z 379 (M+H).

### EXAMPLE 11

Benzene-1,2-disulfonic acid 1-[(4-methoxyphenyl)-amide] 2-pyridin-4-yl amide

4-Aminopyridine (0.17 g, 1.8 mmol) and triethylamine (0.25 mL, 1.8 mmol) were added to a solution of benzene-1,2-disulfonyl chloride (0.5 g, 1.8 mmol) in dichloromethane (10 mL). The mixture was stirred at room temperature for 3 h. Anisidine (0.22 g, 1.8 mmol) and triethylamine (0.25 mL, 1.8 mmol) were added and the mixture stirred a further 15 h at room temperature. The precipitate was removed by filtration and the filtrate concentrated in vacuo. The residue was triturated with methanol and the insoluble material removed by filtration (this material was purified to give Example 12). The filtrate was concentrated under reduced pressure. The residue was chromatographed on silica gel using 10% methanol in dichloromethane as eluent to give benzene-1,2-disulfonic acid 1-[(4-methoxyphenyl)-amide] 2-pyridin-4-yl amide as a white solid (0.07 g, 9%). The major product was the ring closed anisidine product, 2-(4-methoxyphenyl)-benzo[1,3,2]-dithiazole 1,1,3,3-tetraoxide. ¹H NMR (200 MHz, d₆-DMSO) δ 3.66 (s, 3H), 6.80 (d, J=8.7 Hz, 2H), 6.99 (d, J=7.4 Hz, 2H), 7.02 (d, J=8.7 Hz, 2H), 7.56-7.87 (m, 3H), 8.06 (d, J=7.4 Hz, 2H), 8.17 (d, J=6.6 Hz, 1H), 9.30 (br s, 1H); MS (APCI+) m/z 420 (M+H); MS ((APCI−)) m/z 418 (M−H).

### EXAMPLE 12

Benzene-1,2-disulfonic acid bis-[(4-methoxyphenyl)-amide]

The precipitate from example 11 was chromatographed on silica gel with 30% ethyl acetate/petroleum spirit (40-60° C.) as eluent to give benzene-1,2-disulfonic acid bis-[(4-methoxyphenyl)-amide] as a white solid (17 mg, 2%). The major product was the ring closed anisidine product, 2-(4-methoxyphenyl)-benzo[1,3,2]dithiazole 1,1,3,3-tetraoxide. ¹H NMR (200 MHz, d₆-DMSO) δ 3.66 (s, 6H), 6.79 (d, J=9 Hz, 4H), 7.00 (d, J=9 Hz, 4H), 7.66-7.73 (m, 2H), 7.87-7.94 (m, 2H); MS ((APCI−)) m/z 447 (M−H).

### EXAMPLE 13

Thiophene-2-sulfonic acid [2-(4-methoxyphenylsulfamoyl)-phenyl]-amide

2-Amino-N-(4-methoxyphenyl)benzenesulfonamide (0.10 g, 0.36 mmol, preparation is described in Example 3) was added to a solution of 2-thiophenyl-sulfonyl chloride (0.13 g, 0.72 mmol) and triethylamine (0.2 mL, 1.44 mmol) in dichloromethane (5 mL) and stirred at room temperature for 16 h under nitrogen. The solvent was removed in vacuo to give crude product. Chromatography on silica gel using dichloromethane as eluent to give thiophene-2-sulfonic acid [2-(4-methoxy-phenylsulfamoyl)-phenyl]-amide as a light yellow oil (94 mg, 62%).

¹H NMR (200 MHz, CDCl₃) δ 3.80 (s, 3H), 6.61-6.85 (m, 3H), 6.82 (d, J=9.1 Hz, 1H), 7.00-7.10 (m, 1H), 7.05 (d, J=9.0 Hz, 1H), 7.13 (dd, J=5.0, 1.1 Hz, 1H), 7.28-7.38 (m, 1H), 7.54 (dd, J=8.2, 1.5 Hz, 1H), 7.70-7.75 (m, 2H); ¹³C NMR (50.3 MHz, CDCl₃) δ 55.5, 114.4, 117.0, 117.6, 119.5, 126.1, 127.5, 131.3, 132.8, 134.2, 135.4, 135.7, 139.2, 146.1, 160.9; MS (APCI+) m/z 425 (M+H); MS ((APCI−)) m/z 423 (M−H).

### EXAMPLE 14

1,2-Bis(2,4,6-triisopropyl-N-phenyl)-benzenesulfonamide

Triethylamine (2.8 mL, 0.02 mol) and 2,4,6-triisopropyl benzene sulfonyl chloride (3.0 g, 0.01 mol) were added to a stirred solution of 1,2-phenylenediamine (1.1 g, 0.01 mol) in dichlromethane (25 mL). The reaction was stirred at room temperature under nitrogen for 24 h. The solvent was removed in vacuo and the residue chromatographed on silica gel using 15% ethyl acetate/petroleum spirit (40-60° C.) as eluent to give 1,2-bis(2,4,6-triisopropyl-N-phenyl)-benzenesulfonamide as a white solid (0.21 g, 3%).

¹H NMR (200 MHz, CDCl₃) δ 1.14 (d, J=7.5 Hz, 18H), 1.25 (d, J=7.5 Hz, 18H), 2.89 (sep, J=5.5 Hz, 2H), 3.88 (sep, J=5.5 Hz, 4H), 6.85-6.95 (br m, 2H), 6.95-7.06 (br m, 2H), 7.22 (br s, 4H); MS (APCI+) m/z 641 (M+H); MS ((APCI−)) m/z 639 (M−H).

### EXAMPLE 15

2-[Benzyl-(4-methoxyphenyl)-sulfamoyl]-N-pyridin-4-yl-benzamide

4-Aminopyridine (0.14 g, 1.5 mmol) and triethylamine (0.21 mL, 1.5 mmol) were added to a solution of benzyl-(4-methoxyphenyl)amine (0.5 g, 1.2 mmol) in toluene (50 mL). The mixture was heated at reflux for 2 h. The solvent was removed in vacuo to give crude product. Chromatography on silica gel using methanol/dichloromethane (1:9) as eluent gave 2-[benzyl-(4-methoxyphenyl)-sulfamoyl]-N-pyridin-4-yl-benzamide as a white solid (0.37 g, 64%).

¹H NMR (200 MHz, CDCl₃) δ 3.65 (s, 3H), 4.69 (s, 2H), 6.59 (d, J=9 Hz, 2H), 6.91 (d, J=9 Hz, 2H), 7.18 (br m, 5H), 7.40-7.60 (br m, 3H), 7.60-7.75 (br m, 1H), 8.45 (br s, 2H), 8.96 (br s, 1H); MS (ES+) m/z 474 (M+H); MS (ES−) m/z 472 (M−H).

### EXAMPLE 16

2-(4-Methoxyphenyl)-sulfamoyl]-N-pyridin-4-yl-benzamide

Triethylamine (0.10 mL, 0.72 mmol) and 10% palladium on carbon (0.17 g, 0.16 mmol Pd) were added to a solution of N-{2[benzyl-(2,4,6-triisopropylbenzenesulfonyl)-amino]-phenyl}-isonicotinamide (0.17 g, 0.36 mmol, preparation is described in Example 15) in methanol (15 mL). Formic acid (0.07 g, 1.48 mmol) was added and the mixture stirred under a nitrogen atmosphere at 60° C. for 2 h. The reaction mixture was filtered through celite and the filtrate concentrated under reduced pressure. The crude product was redissolved in dichloromethane (20 mL), washed with water (2×15 mL) and dried (MgSO₄). The solvent was removed in vacuo to give crude product which was recrystallised from ethanol to give 2-[4-methoxyphenyl)-sulfamoyl]-N-pyridin-4-yl-benzamide as a white solid (0.08 g, 55%).

¹H NMR (200 MHz, d₆-DMSO) δ 3.70 (s, 3H), 6.82 (d, J=6 Hz, 2H), 7.06 (d, J=6 Hz, 2H), 7.70 (br m, 6H), 8.51 (d, J=5 Hz, 2H), 9.49 (br s, 1H), 10.98 (br s, 1H); MS (APCI+) m/z 384 (M+H); MS ((APCI−)) m/z 382 (M−H).

### EXAMPLE 17

4-Fluoro-N-[2-(3,4,5-trimethoxybenzenesulfonylamino)-phenyl]-benzamide

3,4,5-Trimethoxyaniline (3.76 g, 20.5 mmol) was added to a stirred suspension of 2-nitrobenzenesulfonyl chloride (5.01 g, 22.6 mmol) and triethylamine (2.38 g, 23.6 mmol) in dry dichloromethane (250 mL). Stirring was continued for 16 h under an argon atmosphere. The reaction mixture was washed with H₂O (3×300 mL), dried (MgSO₄) and concentrated in vacuo to give 3,4,5-trimethoxyphenyl-2-nitrobenzenesulfonamide as a dark green solid (5.81 g, 69%).

3,4,5-Trimethoxyphenyl-2-nitrobenzenesulfonamide (5.81 g, 15.7 mmol) was suspended in dry ethanol (200 mL). 10% Palladium on carbon (0.6 g, 0.6 mmol Pd) and glacial acetic acid (1 mL) were added and the mixture stirred at room temperature, under an argon atmosphere for 48 h. The mixture was filtered through glass fibre filter paper (GF/A) and the filtrate concentrated to give 3,4,5-trimethoxyphenyl-2-aminobenzenesulfonamide as a grey/green solid (4.64 g, 87%).

Dry N,N-dimethylformamide (50 μL) was added to a stirred suspension of 4-fluorobenzoic acid (124 mg, 0.89 mmol) and oxallyl chloride (77 μL, 0.87 mmol) in dichloromethane (4 mL) at 0° C., under an argon atmosphere. Reaction was allowed to warm to room temperature and stirred for 45 min. 3,4,5-Trimethoxyphenyl-2-aminobenzenesulfonamide (200 mg, 0.59 mmol) and triethylamine (125 μL, 0.89 mmol) were dissolved in dichloromethane (3 mL) and added to the acid chloride solution. The reaction mixture was stirred at room temperature under an argon atmosphere for 16 h. The precipitate was removed by filtration, and the filtrate concentrated in vacuo. The crude product was suspended in ethyl acetate, filtered, and the filtrate washed with dil. NaHCO₃ (2×10 mL) and water (3×10 mL). The organic layer was dried (MgSO₄) and concentrated in vacuo to give 4-fluoro-N-[2-(3,4,5-trimethoxy-benzenesulfonylamino)-phenyl]-benzamide as a brown solid (137 mg, 50%).

¹H NMR (200 MHz, CDCl₃) δ 3.60 (s, 6H), 3.68 (s, 3H), 6.16 (s, 4H), 7.05-7.24 (m, 4H), 7.62 (t, J=7 Hz, 1H), 7.78-7.92 (m, 3H), 8.51 (d, J=7 Hz, 1H), 9.92 (s, 1H); MS (APCI+) m/z 483 (M+Na); MS ((APCI−)) m/z 459 (M−H).

### EXAMPLE 18

1H-Pyrrole-2-carboxylic acid [2-(3,4,5-trimethoxybenzenesulfonylamino)-phenyl]-benzamide

Dry N,N-dimethylformamide (50 μL) was added to a stirred suspension of pyrrol-2-carboxylic acid (99 mg, 0.87 mmol) and oxallyl chloride (77 μL, 0.87 mmol) in dichloromethane (4 mL) at 0° C., under an argon atmosphere. Reaction was allowed to warm to room temperature and stirred for 45 min. 3,4,5-Trimethoxyphenyl-2-aminobenzenesulfonamide (preparation is described in Example 17) (203 mg, 0.60 mmol) and triethylamine (125 μL, 0.89 mmol) were dissolved in dichloromethane (3 mL) and added to the acid chloride solution. The reaction mixture was stirred at room temperature under an argon atmosphere for 16 h. The precipitate was removed by filtration, and the filtrate concentrated in vacuo. The crude product was suspended in ethyl acetate, filtered, and the filtrate washed with dil. NaHCO₃ (2×10 mL) and water (3×10 mL). The organic layer was dried (MgSO₄) and concentrated in vacuo to give 1H-pyrrole-2-carboxylic acid [2-(3,4,5-trimethoxybenzenesulfonyl-amino)-phenyl]-benzamide as a brown solid (183 mg, 71%).

¹H NMR (200 MHz, CDCl₃) δ 3.40 (s, 6H), 3.60 (s, 3H), 6.15 (s, 1H), 6.29 (m, 1H); 6.75 (m, 1H), 7.00 (m, 1H), 7.14 (t, J=7 Hz, 1H), 7.55 (t, J=7 Hz, 1H), 7.81 (d, J=7 Hz, 1H), 8.41 (d, J=7 Hz, 1H), 9.35 (s, 1H), 9.68 (s, 1H); MS (APCI+) m/z 454 (M+Na); MS ((APCI−)) m/z 430 (M−H).

### EXAMPLE 19

N-[2-(3,4,5-Trimethoxybenzenesulfonylamino)-phenyl]-isonicotinamide

Dry N,N-dimethylformamide (50 μL) was added to a stirred suspension of isonicotinic acid (110 mg, 0.89 mmol) and oxallyl chloride (77 μL, 0.87 mmol) in dichloromethane (4 mL) at 0° C., under an argon atmosphere. Reaction was allowed to warm to room temperature and stirred for 45 min. 3,4,5-Trimethoxyphenyl-2-aminobenzenesulfonamide (preparation is described in Example 17) (201 mg, 0.60 mmol) and triethylamine (125 μL, 0.89 mmol) were dissolved in dichloromethane (3 mL) and added to the acid chloride solution. The reaction mixture was stirred at room temperature under an argon atmosphere for 16 h. The precipitate was removed by filtration, and the filtrate concentrated in vacuo. The crude product was suspended in ethyl acetate, filtered, and the filtrate washed with dil. NaHCO₃ (2×10 mL) and water (3×10 mL). The organic layer was dried (MgSO₄) and concentrated in vacuo. The crude product was triturated with ethyl acetate, dichloromethane, and methanol to give N-[2-(3,4,5-trimethoxybenzenesulfonylamino)-phenyl]-isonicotinamide as a grey/brown solid (92 mg, 35%).

¹H NMR (200 MHz, d₆-DMSO) δ 3.36 (s, 6H), 3.55 (s, 3H), 6.21 (s, 2H), 7.41 (m, 1H), 7.65-7.82 (m, 3H), 7.89 (d, J=6 Hz, 1H), 8.28 (d, J=6 Hz, 1H), 8.85 (d, J=6 Hz, 2H), 10.18 (d, J=6 Hz, 1H); MS (APCI+) m/z 444 (M+H).

### EXAMPLE 20

Rat Aorta Assay for Angiogenesis Inhibition

The rat aorta ring model based on that described by Nicosia and Ottinetti (Nicosia, R. F. et al. Lab. Investigation 63: 115, 1990; Nicosia, R. F. et al. Cell. Dev. Biol. 26: 119-128, 1990), was used throughout the assay.

The agarose was made up as a 1.5% solution in distilled water and brought to the boil to form a clear solution which was poured into sterile 9 cm petri dishes, covered and allowed to cool and set.

Maintaining sterile conditions, agarose rings were obtained by punching two concentric circles, with sterile10 and 17 mm hole punches, respectively, in the agarose gel. Using sterile forceps, the rings are removed and placed, three per well in each of the 6-well plates.

The MEM was prepared according to manufacturer's directions, but before filtering through a 0.22 μm filter, HEPES and L-glutamine were added to give 10 mM and 1 mM concentrations respectively with pH adjusted to 7.4. Eight hundred mL of this medium were filtered through a 0.2 μm filter along with the antibiotics (50 mg/L Gentamycin sulphate and 2.5 mg/L Amphotericin B) and 200 mL of FCS (to give 20%) to yield one litre of medium.

The aorta was removed from a 3-4 month male Copenhagen rat and transferred to a dissecting dish where it was cleaned and carefully stripped of the fibroadipose tissue surrounding it. Rings of 0.5 mm were cut, using a fresh scalpel blade, from the length of the aorta. These were kept under sterile conditions in a biohazard hood where they were washed 12 times with MEM.

Before transferring the aortic rings to the culture plate, the bottom of each agarose well was coated with 150 μL of clotting fibrinogen. Fibrinogen was made up as a 3 mg/mL solution in MEM, while thrombin made up in distilled water to give a concentration of 50 U/mL. The fibrinogen (1 mL) and the thrombin (20 μL) reacted within 30 sec to form a solid gel.

The aortic rings were transferred to the 6-well plates, with one ring placed in the center of each agarose well. Fresh fibrinogen/thrombin was made up as before and 150 μL was used to seal in each aortic ring. The gels were rested for approximately 2 hours before the medium was added.

The test compounds were prepared to give three concentration for testing—4, 20 and 100 μg/mL. The compounds were made up as 6 mg/mL solution in water or DMSO. The test solutions were added to each well with the medium.

Six mL of MEM were carefully added to each of three wells to become the controls. MEM, along with the test compounds were added to the remaining wells and all were covered and transferred to the CO₂ Incubator at 37° C., where they were kept for the next 14 days.

The plates were checked each day, but very little growth was observed in the first 4 days. However, by the fifth day, there were noticeable changes in the tissue. Micro-vessels were seen and scored. We have based the scoring method on that used by Liekens et al. (Liekens, S., et al. Oncol. Res. 9: 173-181, 1997) in which 0 meant no vessels and 10 meant maximum vessels; the score is then converted into a percentage inhibition of vessel growth.

Results

| | | Growth | Growth |
|---|---|---|---|
| | Concentration | Inhibition % | Inhibition % |
| Example | (μg/mL) | (Day) | (Day) |
| 1 | 50 | 100 (7) | 100 (14) |
| | 20 | 98 (7) | 98 (14) |
| | 4 | 100 (7) | 100 (14) |
| 2 | 50 | 70 (5) | |
| | 20 | 90 (5) | |
| | 4 | 50 (5) | |
| 3 | 10 | 100 (5) | 100 12) |
| | 4 | 100 (5) | 100 (12) |
| | 1 | 100 (5) | 100 (12) |
| | 0.5 | 100 (5) | 60 (12) |
| 5 | 10 | 80 (5) | 70 (12) |
| 6 | 10 | 10 (7) | |
| 8 | 10 | 80 (5) | 30 (12) |
| 12 | 10 | | 20 (12) |
| 13 | 10 | 100 (7) | 100 (12) |
| | 4 | 100 (7) | 100 (12) |
| | 1 | 100 (7) | 100 (12) |
| | 0.5 | 90 (7) | 90 (12) |
| 15 | 4 | 90 (7) | |
| 16 | 10 | 70 (7) | 10 (12) |

### EXAMPLE 21

Cytotoxicity Assay

HeLa cervical adenocarcinoma (epithelial) cells were cultured in the presence of 100 μM of test compound for 72 hours and cell viability was measured using the WST-1 tetrazolium salt (cleaved to formazan by mitochondrial respiratory chain enzymes, which are only active in viable cells). The absorbance of the dye solution was measured at 450 nm. Controls included cells alone, 0.05% NP-40 plus cells (100% cytotoxicity control) and each of the compound vehicles alone, DMSO or water at the same final concentration in medium as the compound in test wells. % Cytotoxicity was calculated as follows: % cytotoxicity=100×(1-OD_{test compound}/OD_{cells alone}).

Results

| Example (100 μM) | % Cytotoxicity |
|---|---|
| None | 0 |
| 1 | 94 |
| 3 | 92 |
| 4 | 56 |
| 5 | 91 |
| 8 | 84 |
| Vehicle - DMSO | −19 |
| Vehicle - Water | 13 |

### EXAMPLE 22

Growth Inhibition Assay

HUVEC (1.5×10³) are plated in a 96-well plate in 100 μL of EBM-2 (Clonetic # CC3162). After 24 h (day 0), the test compound (100 μL) is added to each well at twice the desired concentration (5-7 concentration levels) in EBM-2 medium. On day 0, one plate is stained with 0.5% crystal violet in 20% methanol for 10 minutes, rinsed with water, and air-dried. The remaining plates are incubated for 72 h at 37° C. After 72 h, plates are stained with 0.5% crystal violet in 20% methanol, rinsed with water and air-dried. The stain is eluted with 1:1 solution of ethanol:0.1M sodium citrate (including day 0 plate), and absorbance is measured at 540 nm with an ELISA reader (Dynatech Laboratories). Day 0 absorbance is subtracted from the 72 h plates and data is plotted as percentage of control proliferation (vehicle treated cells). IC₅₀ (drug concentration causing 50% inhibition) is calculated from the plotted data.

Results

| Example | IC₅₀ | |
|---|---|---|
| 1 | 2.4 ± 0.5 | μM |
| 3 | 0.29 ± 0.22 | μM |
| 5 | 7.1 ± 2.7 | μM |
| 12 | >75 | μM |
| 13 | 0.28 ± 0.22 | μM |

### EXAMPLE 23

Cord Formation Assay

Matrigel (60 μL of 10 mg/mL) is placed in each well of an ice-cold 96-well plate. The plate is allowed to sit at room temperature for 15 minutes then incubated at 37° C. for 30 minutes to permit the matrigel to polymerize. In the mean time, HUVEC are prepared in EGM-2 (Clonetic # CC3162) at a concentration of 2×10⁵ cells/mL. The test compound is prepared at twice the desired concentration (5 concentration levels) in the same medium. Cells (50 μL) and 2×drug (500 μL) is mixed and 200 μL of this suspension are placed in duplicate on the polymerized matrigel. After 24 h incubation, triplicate pictures are taken for each concentration using a Bioquant Image Analysis system. Drug effect (IC₅₀) is assessed compared to untreated controls by measuring the length of cords formed and number of junctions.

Results

| Example | IC₅₀ | |
|---|---|---|
| 3 | 0.48 ± 0.27 | μM |
| 5 | 36.1 ± 9.4 | μM |
| 12 | >50 | μM |
| 13 | 0.41 ± 0.03 | μM |

### EXAMPLE 24

Cell Migration Assay

Migration is assessed using the 48-well Boyden chamber and 8 μm pore size collagen-coated (10 μg/mL rat tail collagen; Collaborative Laboratories) polycarbonate filters (Osmonics, Inc.). The bottom chamber wells receive 27-29 μL of DMEM medium alone (baseline) or medium containing chemo-attractant (bFGF, VEGF or Swiss 3T3 cell conditioned medium). The top chambers receive 45 μL of HUVEC cell suspension (×10⁶ cells/mL) prepared in DMEM+1% BSA with or without test compound. After 5 h incubation at 37° C., the membrane is rinsed in PBS, fixed and stained in Diff-Quick solutions. The filter is placed on a glass slide with the migrated cells facing down and cells on top are removed using a Kimwipe. The testing is performed in 4-6 replicates and five fields are counted from each well. Negative unstimulated control values are subtracted from stimulated control and drug treated values and data is plotted as mean migrated cell±S.D. IC₅₀ is calculated from the plotted data.

Results

| Example | IC₅₀ | |
|---|---|---|
| 3 | 0.68 ± 0.17 | μM |
| 12 | 2.3 | μM |
| 13 | 0.62 ± 0.25 | μM |

Persons skilled in this art will appreciate that variations and modifications may be made to the invention as broadly described herein, other than those specifically described, without departing from the spirit and scope of the invention. It is to be understood that this invention extends to include all such variations and modifications.

1. A compound of Formula I: 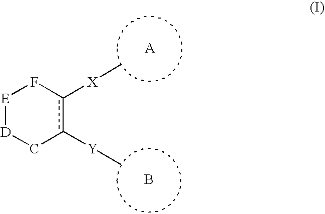
or a pharmaceutically acceptable salt thereof,
wherein:
A and B are each independently selected from the group consisting of
alkyl, alkenyl, alkynyl, arylalkyl, heteroarylalkyl, cycloalkyl, heterocycloalkyl, aryl, and heteroaryl;
in which arylalkyl, heteroarylalkyl, cycloalkyl, heterocycloalkyl, aryl and heteroaryl groups may be connected with another ring through a single bond or fused with at least one other ring, and these rings optionally substituted at one or more positions with:
alkyl, alkoxy, aryl, aryloxy, arylalkyl, arylalkyloxy, cyano, halogen, nitro, oxo, thiono, or CHₙXₘ (where X is halogen, m is 1 to 3, and n is 3-m);
S(O)R, or S(O)₂R, (wherein R is selected from the group consisting of hydroxy, alkyl, alkoxy, aryl, aryloxy, arylalkyl, and arylalkyloxy);
C(O)R, NHC(O)R, or (CH₂)ₙC(O)OR, (wherein R is selected from the group consisting of hydrogen, hydroxy, alkyl, alkoxy, aryl, aryloxy, arylalkyl, and arylalkyloxy, and n is 0-11);
S(O)₂OR, OR, SR, B(OR)₂, PR₃, P(O)(OR)₂, OP(O)(OR)₂, or ═NOR, (wherein R is selected from the group consisting of hydrogen, alkyl, aryl, and arylalkyl); or
NRR′, NRS(O)₂R′, SO₂NRR′, or CONRR′, (wherein R is selected from the group consisting of hydrogen, alkyl, aryl, and arylalkyl, and R′ is selected from the group consisting of hydrogen, hydroxy, alkyl, alkoxy, aryl, aryloxy, arylalkyl, and arylalkyloxy) and
(i) where A is an alkyl, alkenyl or alkynyl group, B is an arylalkyl, heteroarylalkyl, cycloalkyl, heterocycloalkyl, aryl, or heteroaryl group;
(ii) where B is an alkyl, alkenyl or alkynyl group, A is an arylalkyl, heteroarylalkyl, cycloalkyl, heterocycloalkyl, aryl, or heteroaryl group;
and wherein:
the dotted line bonds of the central ring indicate the possibility of a double bond or a delocalised aromatic bond;
C is CR¹, nitrogen, oxygen, or sulfur;
D is CR², nitrogen, oxygen, or sulfur;
E is CR³, nitrogen, oxygen, or sulfur;
F is CR⁴, nitrogen, oxygen, sulfur, or nothing;
provided that at least one of C, D, E, or F is CR; and R¹, R², R³, R⁴ are each independently selected from:
hydrogen, alkyl, alkenyl, alkynyl, alkoxy, aryl, aryloxy, arylalkyl, arylalkyloxy, cycloalkyl, cyano, halogen, heteroaryl, nitro, or CHₙXₘ (where X is halogen, m is 1 to 3, and n is 3-m);
S(O)R, or S(O)₂R, (wherein R is selected from the group consisting of hydroxy, alkyl, alkoxy, aryl, aryloxy, arylalkyl, and arylalkyloxy);
C(O)R, NHC(O)R, or (CH₂)ₙC(O)OR, (wherein R is selected from the group consisting of hydrogen, hydroxy, alkyl, alkoxy, aryl, aryloxy, arylalkyl, and arylalkyloxy, and n is 0-11);
S(O)₂OR, OR, SR, B(OR)₂, PR₃, P(O) (OR)₂, OP(O) (OR)₂, or —NOR, (wherein R is selected from the group consisting of hydrogen, alkyl, aryl, and arylalkyl);
NRR′, NRS(O)₂R′, SO₂NRR′, or CONRR′, (wherein R is selected from the group consisting of hydrogen, alkyl, aryl, and arylalkyl, and R′ is selected from the group consisting of hydrogen, hydroxy, alkyl, alkoxy, aryl, aryloxy, arylalkyl, and arylalkyloxy);
or one of R¹ and R², or R² and R³, or R³ and R⁴ are taken together with the carbon atoms to which they are attached to form a carbocycle or heterocycle;
and wherein:
X and Y are linker groups each selected independently from the group consisting of: SO₂NR, NRSO₂, C(O)NR, NRC(O), C(S)NR, NRC(S), NRC(O)O, NRC(S)S, C(O)O, OC(O), S(O)₂O, OSO₂, SO₂, OS(O), OSO₂NR, NRS(O)₂NR′, C(S)SSNR, NRSSC(S), P(O) (OR)NR′, NRP(O) (OR′), NRP(O)(OR′)O, CR═CR′, NRC(O)NR′, NR, C═NO—, —ON═C, C═N, N═C, N═N (→O)—, N (→O)=N, N═N, and a direct bond; where R and R¹ are each selected independently from the group consisting of hydrogen, alkyl, alkenyl, alkynyl, cycloalkyl, aryl, arylalkyl, acyl, alkoxyacyl, aryloxyacyl, or aminoacyl; and
(i) where X is NRSO₂, Y is not NRC(O), NRC(S), NR, NRC(O)O or NRC(O)NR;
(ii) where Y is NRSO₂, X is not NRC(O), NRC(S), NR, NRC(O)O or NRC(O)NR;
(iii) where X is a direct bond, Y not a direct bond; and
(iv) where Y is a direct bond, X is not a direct bond.

2. A compound of Formula I as defined in claim 1, wherein X is SO₂NR⁵, Y is CONR⁶, and R⁵ and R⁶ are each independently selected from the group consisting of H, alkyl, and aryl.

3. A compound of Formula I as defined in claim 1, wherein X is SO₂NR⁵, Y is NR⁶CO, and R⁵ and R⁶ are as defined in claim 2.

4. A compound of Formula I as defined in claim 1, wherein X is NR⁵SO₂, Y is CONR⁶, and R⁵ and R⁶ are as defined in claim 2.

5. A compound of Formula I as defined in claim 1, wherein X is SO₂NR⁵, Y is SO₂NR⁶, and R⁵ and R⁶ are as defined in claim 2.

6. A compound of Formula I as defined in claim 1, wherein X is SO₂NR⁵, Y is NR⁶SO₂, and R⁵ and R⁶ are as defined in claim 2.

7. A compound of Formula I as defined in claim 1, wherein X is CONR⁵, Y is CONR⁶, and R⁵ and R⁶ are as defined in claim 2.

8. A compound of Formula I as defined in claim 1, wherein X is CONR⁵, Y is NR⁶CO, and R⁵ and R⁶ are as defined in claim 2.

9. A compound of Formula I as defined in claim 1, wherein X is NR⁵SO₂, Y is NR⁶SO₂, and R⁵ and R⁶ are as defined in claim 2.

10. A compound of Formula I as defined in claim 1, wherein X is NR⁵CO, Y is NR⁶CO, and R⁵ and R⁶ are as defined in claim 2.

11. A compound of Formula I as defined in claim 1, wherein X is NR⁵CONR⁶, Y is NR⁷CONR⁸, R⁵ and R⁶ are as defined in claim 2, and wherein R⁷ and R⁸ are each independently selected from the group consisting of H, alkyl, and aryl.

12. A compound of Formula I as defined in claim 1, wherein X is SO₂NR⁵, Y is NR⁶CS, and R⁵ and R⁶ are as defined in claim 2.

13. A compound of Formula I as defined in claim 1, wherein X is SO₂NR⁵, Y is NR⁶CO₂, and R⁵ and R⁶ are as defined in claim 2.

14. A compound of Formula I as defined in claim 1, wherein X is SO₂NR⁵, Y is NR 6, and R⁵ and R⁶ as defined in claim 2.

15. A compound of Formula I as defined in claim 1, wherein X is SO₂NR⁵, Y is NR⁶P(O)R⁷, R⁵ and R⁶ are as defined in claim 2, and wherein R⁷ is selected from the group consisting of H, alkyl, aryl, alkoxy, and aryloxy.

16. A compound of Formula I as defined in claim 1, wherein X is SO₂NR⁵, Y is N═CH, and R⁵ is as defined in claim 2.

17. A compound of Formula I as defined in claim 1, wherein X is SO₂O, Y is CONR⁵, and R⁵ is as defined in claim 2.

18. A compound of Formula I as defined in claim 1, wherein X is SO₂O, Y is NR⁵CO, and R⁵ is as defined in claim 2.

19. A compound of Formula I as defined in claim 1, wherein X is OSO₂, Y is CONR⁵, and R⁵ is as defined in claim 2.

20. A compound of Formula I as defined in claim 1, wherein X is SO₂O, Y is SO₂O.

21. A compound of Formula I as defined in claim 1, wherein X is SO₂O, Y is OSO₂.

22. The compound of Formula I as defined in claim 1, wherein A and B are each selected independently from the group consisting of pyrrolidine, piperidine, piperazine, morphonline, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene, optionally substituted at one or more positions with alkyl, alkoxy, aryl, aryloxy, alkaryl, alkaryloxy, halogen, trihalomethyl, oxo, ═S, S(O)R, SO₂NRR′, S(O)₂OR, SR, B(OR)₂, PR₃, P(O)(OR)₂, OP(O)(OR)₂, NO₂, NRR′, N(O)R, OR, CN, C(O)R, NHC(O)R, (CH₂)ₙCO₂R, and CONRR′, wherein R and R′ are each independently selected from the group consisting of H, alkyl, alkoxy, aryl, aryloxy, arylalkyl, and arylalkyoxy; and n is 0-11.

23. The compound of Formula I as defined in claim 1, wherein the compound is selected from N-(2,6,-Diisopropylphenyl)-2-(2,6-diisopropylphenylsulfamoyl)-benzamide, N-phenyl-2-phenylsulfamoylbenzamide, N-[2-(4-methoxyphenylsulfamoyl)-phenyl]-isonicotinamide, N-[2-(4-methoxyphenylsulfamoyl)-phenyl]-4-nitro-benzamide, N-[2-(4-methoxyphenylsulfamoyl)-phenyl]-4-fluorobenzamide, N,N′-bis-(2,6-diisopropyl-phenyl)-phthalamide, 1-m-tolyl-3-[4-(3-m-tolyl-ureido)-pyridin-3-yl]-urea, 2-(4-methoxybenzenesulfonylamino)-N-pyridin-4-yl-benzamide, 2-(4-methoxy-benzamido)-N-pyridin-4-ylbenzamide, [2-(4-methoxyphenyl-sulfamoyl)-phenyl]-carbamic acid tert-butyl ester, benzene-1,2-disulfonic acid 1-[(4-methoxyphenyl)-amide] 2-pyridin-4-yl amide, benzene-1,2-disulfonic acid bis-[(4-methoxyphenyl)-amide], thiophene-2-sulfonic acid [2-(4-methoxyphenyl-sulfamoyl)-phenyl]-amide, 1,2-bis (2,4,6-triisopropyl-N-phenyl)-benzenesulfonamide, 2-[benzyl-(4-methoxyphenyl)-sulfamoyl]-N-pyridin-4-yl-benzamide, 2-(4-methoxyphenyl)-sulfamoyl]-N-pyridin-4-yl-benzamide, 4-fluoro-N-[2-(3,4,5-trimethoxybenzenesulfonylamino)-phenyl]-benzamide, 1H-pyrrole-2-carboxylic acid [2-(3,4,5-trimethoxybenzenesulfonyl-amino)-phenyl]-benzamide, and N-[2-(3,4,5-trimethoxybenzenesulfonylamino)-phenyl]-isonicotinamide.

24. A compound according to claim 2, wherein the compound is N-2,6,-diisopropylphenyl)-2-(2,6-diisopropylphenylsulfamoyl)-benzamide.

25. A compound according to claim 2, wherein the compound is 2-(4-methoxyphenyl)-sulfamoyl]-N-pyridin-4-yl-benzamide.

26. A compound according to claim 6, wherein the compound is thiophene-2-sulfonic acid [2-(4-methoxyphenylsulfamoyl)-phenyl]-amide.

27. A compound according to claim 3, wherein the compound is N-[2-(4-methoxyphenylsulfamoyl)-phenyl]-isonicotinamide.

28. A pharmaceutical composition comprising therapeutically effective amount of a compound according to any one of claims 1 to 27, or a pharmaceutical acceptable salt thereof, together with a pharmaceutically acceptable carrier or excipient.

29. A method for preventive and/or therapeutic treatment of a disease or disorder arising from abnormal or inappropriate cell proliferation, comprising administration to a subject in need thereof of a therapeutically effective amount of a compound according to any one of claims 1 to 27, or a pharmaceutically acceptable salt thereof.

30. The method of claim 29, wherein said treatment is treatment of a neoplastic disease or neoplastic dependent disorder, including tumour growth, tumour metastasis and associated angiogenesis.

31. The method of claim 29 or claim 30, wherein said administration is in conjunction with another preventative or therapeutic treatment of a disease or disorder arising from abnormal or inappropriate cell proliferation, including angiogenesis.

32. The method of any one of claims 29 to 31, wherein said subject is a human.

33. Use of a compound according to any one of claims 1 to 27, or a pharmaceutically acceptable salt thereof, in the manufacture of a composition for preventative and/or therapeutic treatment of a disease or disorder arising from abnormal or inappropriate cell proliferation.
